(19) United States
(12) Patent Application Publication
(10) Pub. No.: US 2005/0080417 A1
(43) Pub. Date: Apr. 14, 2005

(54) Occipital fixation device

(75) Inventors: Faure Alexis, Nantes (FR); Alexandre Deborah, Lille (FR); Rokegem Pascal, Arras (FR); Leroy Eric, Saint-Nicolas Les Arras (FR)

Correspondence Address: YOUNG & THOMPSON, 745 SOUTH 23RD STREET, 2ND FLOOR, ARLINGTON, VA, 22202, US

(73) Assignee: EUROSURGICAL SA, BEAURAINS CEDEX (FR)

(21) Appl. No.: 10/856,768

(22) Filed: Jun. 1, 2004

(30) Foreign Application Priority Data
Oct. 14, 2003 (FR) 03 11982

(51) Int. Cl. A61B 17/56

(52) U.S. Cl. 606/61

(57) ABSTRACT

The occipital fixation device according to the present invention comprises a first element (10) that is comprised of fixation means for immobilizing in translation and in rotation in a connection rod (5) and engagement means (19, 20) for bone anchoring in the occipital bone (8) and a second element (18) sliding in the first and comprising engagement means (26) supplementing the engagement means (19, 20) of the first element (10) to obtain the bone anchoring of the fixation device (7) in the occipital bone (8).

The present invention relates to an occipital hook fixation device supplementing the range of implants allowing the posterior fixation of cervical vertebrae from a vertebral column.

Fixation devices such as occipital plates are known that are designed to cooperate with posterior cervical instrumentation. The occipital plate presents a curved or banana-shaped profile turned to the left or right according to its fixation position on the occipital bone.

The occipital plate cooperates through a connection device with the cervical instrumentation and more particularly with the connection rod connecting the assembly of bone anchoring devices.

The plate is pierced by a certain number of holes each designed to receive a bone anchoring screw to be anchored in the occipital bone.

The occipital fixation device according to the present invention is provided to cooperate with the connection rod of a spinal device comprising bone anchoring elements that are fixed in the vertebral bodies of a vertebral column.

The occipital hook fixation device according to the present invention comprises a first element that is comprised of fixation means for the immobilization in translation and in rotation of the connection rod and engagement means for the bone anchoring in the occipital bone and a second element sliding in the first which comprises engagement means supplementing the engagement means of the first element to obtain the bone anchoring of the fixation device in the occipital bone.

The occipital hook fixation device according to the present invention comprises a first element provided with fixation means for immobilizing in translation and in rotation the connection rod, these means comprised of a cylindrical body presenting a threaded external profile, an oblong-shaped central housing which cuts, according to a vertical direction, the cylindrical body into two distinct parts for receiving the connection rod, and a retaining nut allowing the connection rod to be immobilized in the first element.

The occipital hook fixation device according to the present invention comprises a first element comprising engagement means that are comprised in the extension of the cylindrical body of a disk, on both sides of a central groove, that is formed as an integral unit with two segments presenting a hook-shaped profile.

The occipital hook fixation device according to the present invention comprises a first element comprising a central groove that is open in its upper part and directed according to a direction perpendicular to that of the oblong housing provided in the cylindrical body.

The occipital hook fixation device according to the present invention comprises a first element comprising a central groove that presents a T-shaped or dovetail-shaped profile comprising in its upper part and on both sides of the sliding axis longitudinal stops allowing the second element in a complementary shape to be held according to a vertical direction.

The occipital hook fixation device according to the present invention comprises a first element comprising hook segments that are disposed in parallel planes separated by a distance whose dimensions are similar to those separating the longitudinal stops of the groove.

The occipital hook fixation device according to the present invention comprises a first element comprising hook segments that comprise engagement parts disposed in parallel planes above the central groove and directed in the same direction.

The occipital hook fixation device according to the present invention comprises a second element that is comprised of an elongated body in which a longitudinal slot is provided on its entire length delimiting two parallel cross members, said elongated body forming an integral unit in its upper part and according to a vertical direction with a segment presenting a hook-shaped engagement part that is disposed above the horizontal cross members.

The occipital hook fixation device according to the present invention comprises a second element comprising a hook segment that is laterally offset from the center of the elongated body.

The occipital hook fixation device according to the present invention comprises a second element comprising a hook segment presenting a thickness that is less than the distance separating the hook segments of the first element.

The occipital hook fixation device according to the present invention comprises a second element comprising an elongated body whose longitudinal slot is extended within and on a part of the height of the hook segment, allowing a certain elasticity to be given between the two cross members of said elongated body.

The occipital hook fixation device according to the present invention comprises a second element comprising cross members presenting an external profile that is complementary to that of the groove provided in the disk of the first element in order to allow the second element to slide in the first.

The description that will follow with regards to the attached designs, given by way of non-limiting examples, will allow the invention, the characteristics that the invention presents and the advantages that the invention is likely to bring to be better understood:

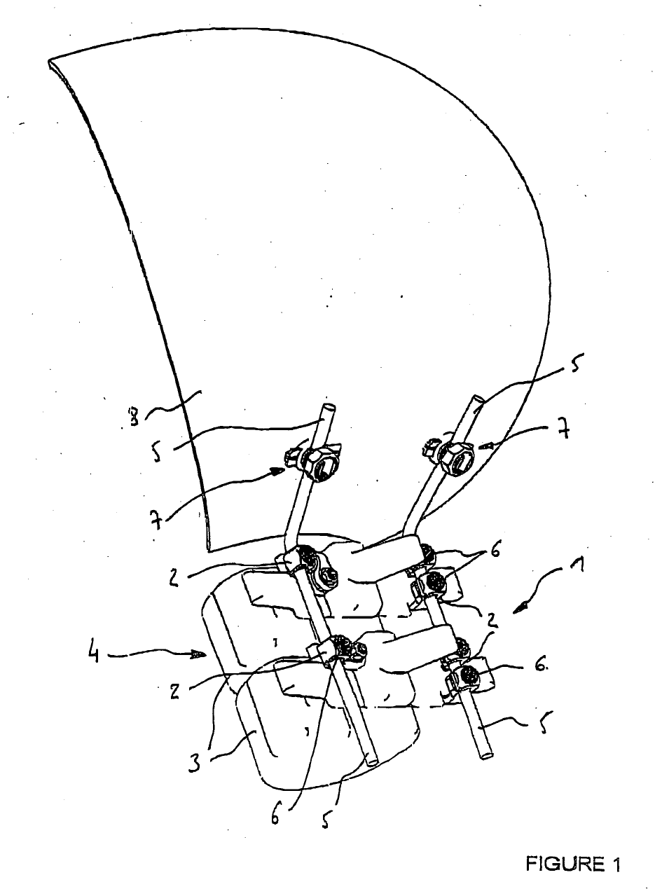

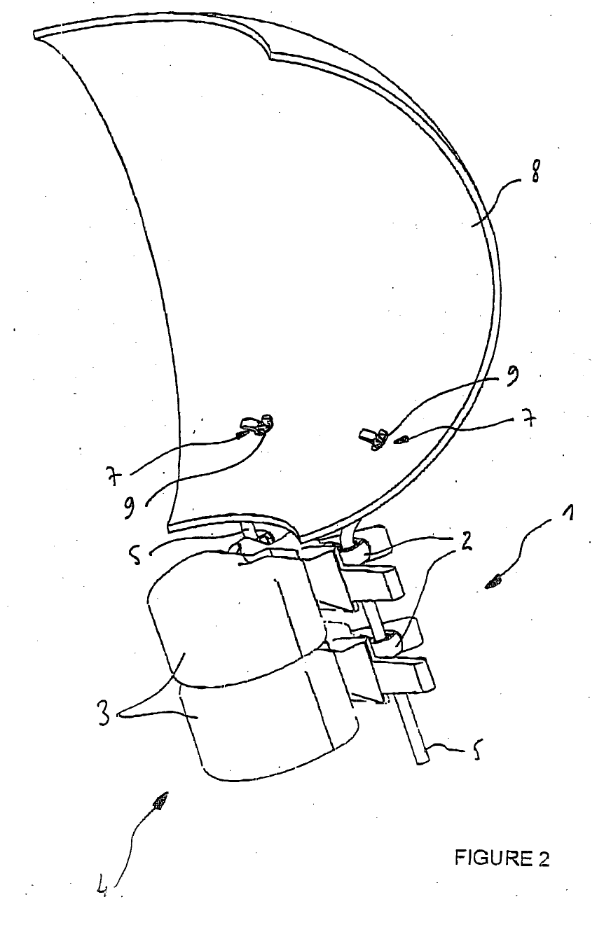

FIGS. 1 and 2 are perspective views illustrating the mounting of a spinal device comprising an occipital hook device according to the present invention on a vertebral column and more particularly at the occipital bone.

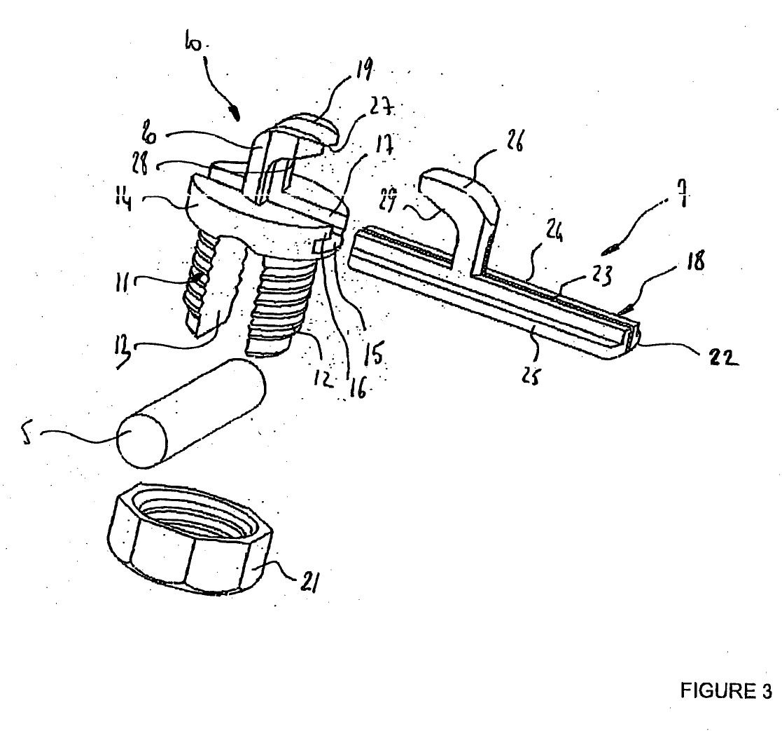

FIG. 3 is an exploded perspective view showing the occipital hook device according to the present invention.

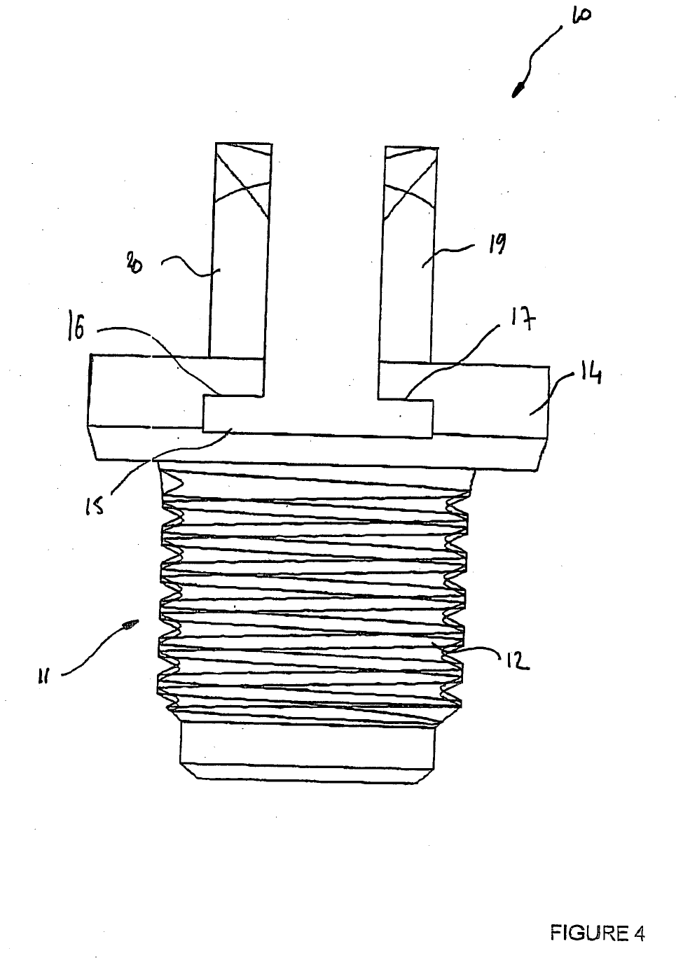

FIG. 4 is a view representing a first hook element of the occipital device according to the present invention.

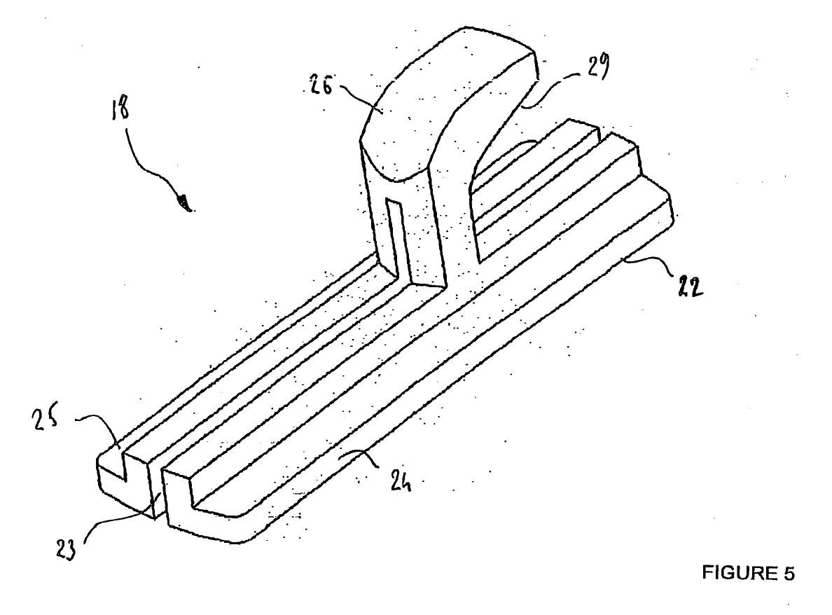

FIG. 5 is a view illustrating a second hook element of the occipital device according to the present invention.

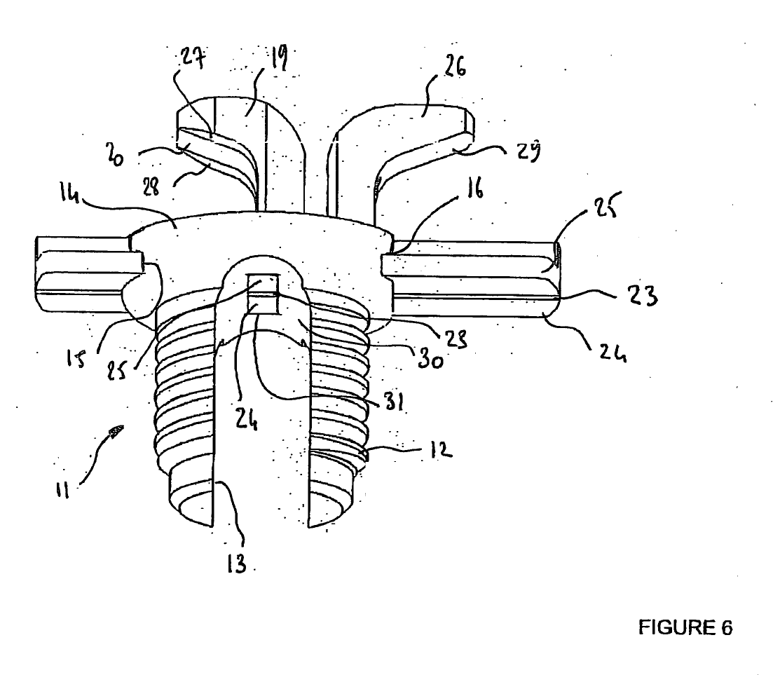

FIG. 6 is a perspective view representing the assembly of the first and second hook elements of the occipital device according to the present invention.

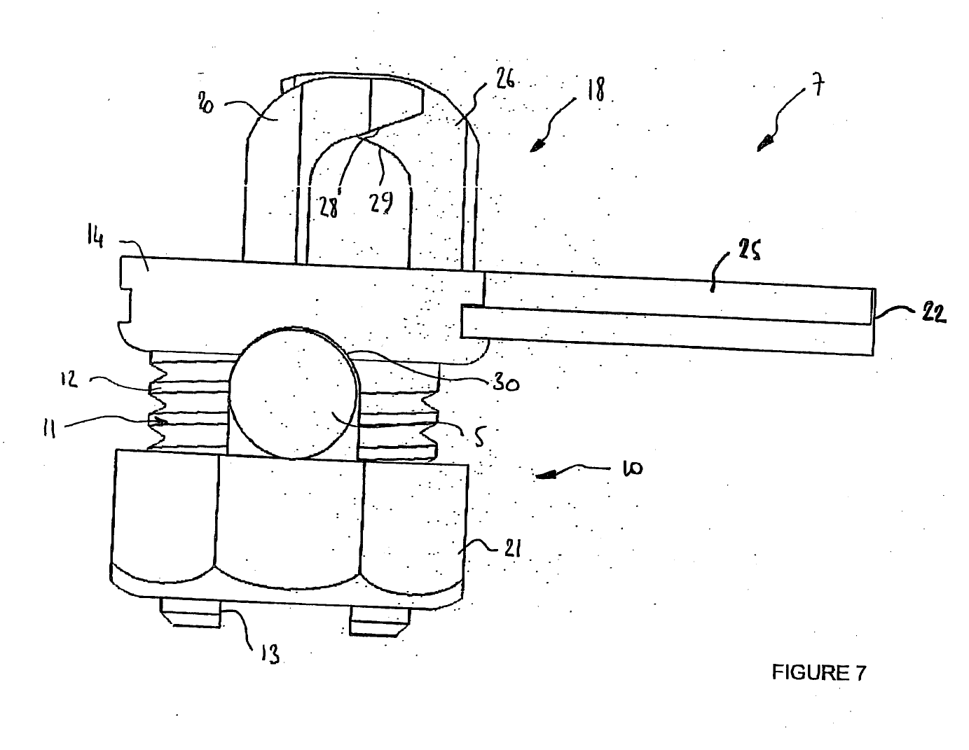

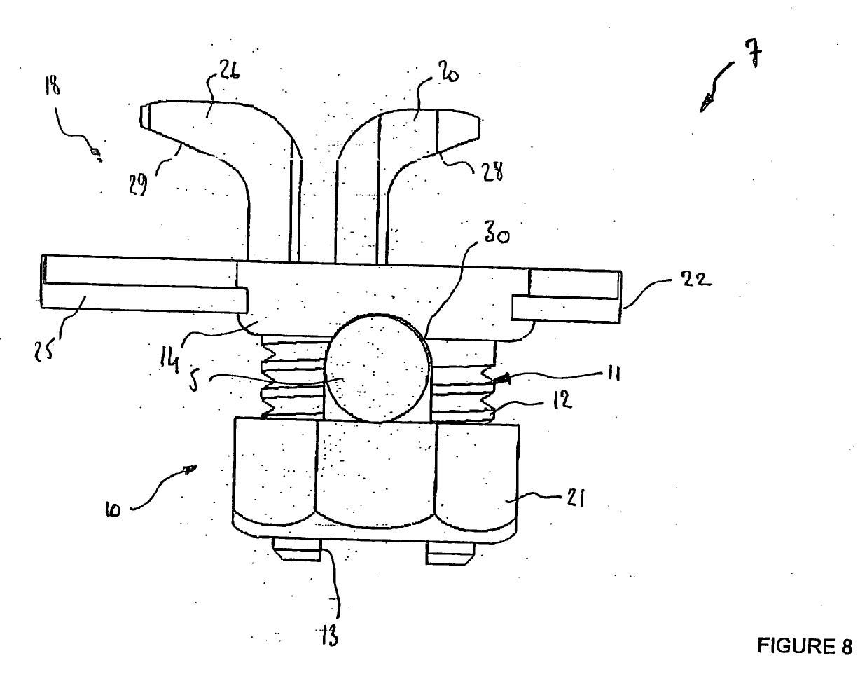

FIGS. 7 and 8 are views showing the mounting of the occipital hook device and more particularly the capability of the second hook element to slide with relation to the first hook element.

FIGS. 1 and 2 show a spinal device 1 comprising bone anchoring elements 2 that are fixed in the vertebral bodies 3 of a vertebral column 4 and interconnected by a connection rod 5.

Each connection rod 5 is immobilized in translation and in rotation in the corresponding anchoring elements 2, by means of a tightening device 6.

The spinal device 1 comprises hook fixation devices 7 allowing each connection rod 5 to be held after piercing holes 9 in the occipital bone 8.

FIGS. 3 to 6 represent different elements comprising the hook fixation device 7 for holding each connection rod 5 at the occipital bone 8.

The fixation device 7 comprises a first hook element 10 comprised of a cylindrical body 11 presenting a threaded external profile 12 and a central oblong-shaped housing 13 cutting the cylindrical body according to a vertical direction into two distinct parts for receiving the connection rod 5 of the spinal device 1.

The cylindrical body 11 forms an integral unit at one of its extremities with a disk 14 crossed by a central groove 15 open in its upper part and directed according to a direction perpendicular to that of the oblong housing 13.

The central groove 15 presents a T-shaped or dovetail-shaped profile comprising in its upper part and on both sides of the sliding axis longitudinal stops 16, 17 allowing a second hook element 18 in a complementary shape to be held according to a vertical direction.

The disk 14 is formed as an integral unit with both sides of the groove 15 and according to a vertical direction opposite from that of the cylindrical body 11 of two segments 19, 20, presenting a hook shaped profile.

The hook segments 19, 20 are disposed in parallel planes separated by a distance whose dimensions are similar to those separating the longitudinal stops 16, 17 of the groove 15.

The engagement parts 27, 28 of each segment 19, 20 are disposed in parallel planes above the central groove 15 and directed in the same direction.

The threaded external profile 12 of the cylindrical body 11 is provided to cooperate with a nut 21 allowing the connection rod 5 to be immobilized within the housing 13 and against its bottom 30 in the shape of a cylinder portion or a U.

The bottom 30 of the housing 13 is pierced by an opening or window 31 communicating with the central groove 15 of the disk 14 (FIG. 6). This opening is obtained by machining the housing 13 and more particularly its bottom 30 wherein the radius of curvature exceeds the bottom of the groove 15 of the disk 14 in order that the latter communicates with the housing 13.

The fixation device 7 comprises a second hook element 18 comprised of an elongated body 22 in which a longitudinal slot 23 is provided on its entire length delimiting the two parallel cross members 24, 25.

The elongated body 22 and more particularly the cross members 24, 25 present an external profile that is complementary to that of the groove 15 provided in the disk 14 of the first hook element 10 in order to allow the second hook element 18 to slide in the first.

The elongated body 22 forms an integral unit in its upper part and according to a vertical direction with a segment 28 presenting a hook-shaped profile wherein the engagement part 29 is disposed above the horizontal cross members 24, 25.

It should be noted that the longitudinal slot 23 is extended within and on a part of the height of the segment 26 allowing a certain elasticity to be given between the two cross members 24, 25 of the elongated body 22.

Also, the segment 26 is laterally offset from the center of the elongated body 22 forming the second hook element 18.

It should be noted that the hook segment 26 of the second element 18 presents a thickness or a width that is less than the distance separating the hook segments 19, 20 of the first element 10.

FIGS. 7 and 8 show the mounting of the fixation device 7 on the connection rod 5 and the sliding of the first and second hook elements 10, 18 with relation to each other.

The fixation device 7 is assembled so that the elongated body 22 of the second hook element 18 slides in the central groove 15 of the first hook element 10.

The hook segment 26 of the second hook element 18 is positioned with relation to the hook segments 19, 20 of the first hook element 10 so that the point of the engagement part 29 of the hook segment 26 does not exceed the vertical part of the hook segments 19, 20 (FIG. 7).

In this position, the engagement parts 27, 28 and 29 of each hook segment 19, 20 and 26 are protected by the vertical part of the other segment.

Also, this position of some hook segments 19, 20 and 26 with relation to the others allows the latter to be introduced in the corresponding hole 9 provided in the occipital bone 8.

Then, through an adapted surgical instrument, the first and second hook elements 10, 18 are displaced one with relation to the other until the engagement parts 27, 28 and 29 of each hook segment 19, 20 and 26 are anchored in the part or on the bone surface and inside the occipital bone bordering the corresponding hole 9 (FIGS. 2 and 8).

It should be noted that the engagement parts 27, 28 and 29 of each hook segment 19, 20 and 26 present an inclined internal profile that improves the anchoring of each hook segment in the bone part of the occipital bone 8.

On the other hand, the introduction of the second hook element 18 within the groove 15 of the first hook element 10 is obtained by elastically deforming the parallel cross members 24, 25 of the elongated body 22 comprising a primary stabilization of the engagement parts 27, 28 and 29 of each hook segment 19, 20 and 26 in the hole 9.

It should be noted that the elongated body 22 of the second hook element 18 crosses the opening or the window 31 provided in the bottom 30 of the housing 13 (FIG. 6).

Once the first and second hook elements 10, 18 of the fixation device 7 are fixed in the corresponding hole 9, the surgeon presents after deformation a connection rod 5 to be introduced within the housing 13 of the first hook element 10.

When the corresponding connection rod 5 is introduced in the housing 13, the latter is immobilized in translation and in rotation through a nut 21 that is screwed on the cylindrical body 11 of the first hook element 10.

The tightening of the nut 21 allows the rod 5 to be immobilized in the bottom 30 of the housing 13 simultaneously leading to the immobilization of the parallel cross members 24, 25 of the elongated body 22 in the groove 15.

In fact, when the connection rod 5 is immobilized, it comes into tight contact against the cross members 24, 25, immobilizing the latter in translation within the groove 15 due to the opening or window 31 that allows communication between the bottom of the groove 15 and that 30 of the housing 13 to be achieved.

In addition, it must be understood that the previous description was given only by way of example and does not in any way limit the scope of the invention wherein details of the executions described may be replaced by any other equivalent without any departure from this scope.

1. An occipital fixation device for a spinal device (1) comprising bone anchoring elements (2) that are fixed in the vertebral bodies (3) of a vertebral column (4) and interconnected through a connection rod (5), characterized in that the device comprises a first element (10) that is comprised of fixation means for immobilizing in translation and in rotation the connection rod (5) and engagement means (19, 20) for the bone anchoring in the occipital bone (8) and a second element (18) sliding in the first that comprises engagement means (26) supplementing the engagement means (19, 20) of the first element (10) to obtain bone anchoring of the fixation device (7) in the occipital bone (8).

2. The occipital fixation device according to claim 1, characterized in that the first element (10) comprises fixation means for the immobilization in translation and in rotation of the connection rod (5) that are comprised of a cylindrical body (11) presenting a threaded external profile (12), an oblong-shaped central housing (13) cutting the cylindrical body into two distinct parts according to a vertical direction for receiving the connection rod (5) and a retaining nut (21) allowing the connection rod (5) to be immobilized in the first element (10).

3. The occipital fixation device according to claim 2, characterized in that the first element (10) comprises engagement means that are comprised in the extension of the cylindrical body (11) of a disk (14) that forms an integral unit on both sides of a central groove (15) with two segments (19, 20) presenting a hook-shaped profile.

4. The occipital fixation device according to claim 3, characterized in that the central groove (15) is open in its upper part and directed according to a direction perpendicular to that of the oblong housing (13) provided in the cylindrical body (11).

5. The occipital fixation device according to claim 3, characterized in that the central groove (15) presents a T-shaped or dovetail-shaped profile comprising in its upper part and on both sides of the sliding axis longitudinal stops (16, 17) allowing the second element (18) in a complementary shape to be held according to a vertical direction.

6. The occipital fixation device according to claim 3, characterized in that the hook segments (19, 20) are disposed in parallel planes separated by a distance whose dimensions are similar to those separating the longitudinal stops (16, 17) of the groove (15).

7. The occipital fixation device according to claim 3, characterized in that the hook segments (19, 20) comprise engagement parts (27, 28) that are disposed in parallel planes above the central groove (15) and directed in the same direction.

8. The occipital fixation device according to claim 1, characterized in that the second element (18) is comprised of an elongated body (22) in which a longitudinal slot (23) is provided on its entire length delimiting two parallel cross members (24, 25), said elongated body (22) forming an integral unit in its upper part and according to a vertical direction with a segment (26) presenting a hook-shaped engagement part (29) that is disposed above the horizontal cross members (24, 25).

9. The occipital fixation device according to claim 8, characterized in that the hook segment (26) is laterally offset from the center of the elongated body (22).

10. The occipital fixation device according to claim 8, characterized in that the hook segment (26) of the second element (18) presents a thickness that is less than the distance separating the hook segments (19, 20) of the first element (10).

11. The occipital fixation device according to claim 8, characterized in that the longitudinal slot (23) is extended within and on a part of the height of the hook segment (26) allowing a certain elasticity to be given between the two cross members (24, 25) of the elongated body (22).

12. The occipital fixation device according to claim 8, characterized in that the cross members (24, 25) of the elongated body (22) present an external profile that is complementary to that of the groove (15) provided in the disk (14) of the first element (10) in order to allow the second element (18) to slide in the first.

13. The occipital fixation device according to claim 2, characterized in that the housing (13) comprises a bottom (30) in the shape of a cylinder portion comprising an opening or window (31) opening in the groove (15) of the disk (14).
